(19) United States
(12) Patent Application Publication
(10) Pub. No.: US 2008/0207572 A1
(43) Pub. Date: Aug. 28, 2008

(54) Use of Dual C-Kit/Fgfr3 Inhibitors for Treating Multiple Myeloma

(75) Inventors: Alain Moussy, Paris (FR); Jean-Pierre Kinet, Lexington, MA (US)

Correspondence Address: FOLEY AND LARDNER LLP;SUITE 500, 3000 K STREET NW, WASHINGTON, DC, 20007, US

(73) Assignee: AB Science

(21) Appl. No.: 11/995,592

(22) PCT Filed: Jul. 13, 2006
(86) PCT No.: PCT/IB06/03111

§ 371 Date: Jan. 14, 2008

Related U.S. Application Data
(60) Provisional application No. 60/698,937, filed on Jul. 14, 2005.

(51) Int. Cl. A61K 31/426; A61K 31/421; A61K 31/496; A61K 31/4439; A61K 31/573; A61P 27/02

(52) U.S. Cl. 514/171; 514/370; 514/377; 514/253.1; 514/340; 514/342

(57) ABSTRACT

The present invention relates to a method for treating Multiple Myeloma (MM), FGFR3+ myeloma, especially relapsed or refractory multiple myeloma (4/14) expressing FGFR3, comprising administering a dual C-KIT/FGFR3 inhibitor, such as 2-aminoarylthiazoles and 2-aminoaryloxazoles.

The present invention relates to a method for treating Multiple Myeloma (MM), FGFR3+ myeloma, especially relapsed or refractory multiple myeloma (4/14) expressing FGFR3, comprising administering a dual C-KIT/FGFR3 inhibitor, such as 2-aminoarylthiazoles and 2-aminoaryloxazoles.

Multiple myeloma (also known as myeloma or plasma cell myeloma) is a progressive hematologic disease, characterized by excessive numbers of abnormal plasma cells in the bone marrow and overproduction of intact monoclonal immunoglobulin. There are about 85,000 new cases/year worldwide (Globocan 2002). Myeloma plasma cells have specific adhesion molecules on their surface allowing them to attach to bone marrow stromal cells. The interaction of cytokines (such as interleukin 6, receptor of activation of NF KB (RANK) ligand and tumor necrosis factor (TNF)), stimulate the growth of myeloma cells and inhibit apoptosis, leading to proliferation of myeloma cells and ultimately resulting in bone destruction. As tumors grow, the myeloma cells spread into the cavities of all the large bones of the body, forming multiple small lesions. Myeloma cells are identical and produce the same immunoglobulin protein, called monoclonal (M) protein or paraprotein, in large quantities. Although the specific M protein varies vary from patient to patient, it is always exactly the same in any one patient.

Multiple myeloma represents approximately 1% of all cancers (the second most common haematological malignancy) and 2% of all cancer deaths. Proliferation of plasma cells is localized (“myelomas”) and characterized by massive localized bone destructions that are a hall mark of the disease and are associated with excuriating pain and bone fractures. Also, hypercalcemia, anemia, renal damage, increased susceptibility to bacterial infection due to impaired production of normal immunoglobulin are common clinical manifestations of multiple myeloma.

The diagnosis of MM is confirmed with the:
detection of an M-protein in the serum or urine.
detection of more than 10% plasma cells on a bone marrow examination.
detection of lytic bone lesions or generalised osteoporosis in skeletal X-rays
presence of soft tissue plasmacytoma

MM is staged by estimating the myeloma tumour cell mass on the basis of the amount of monoclonal (or myeloma) protein (M-protein) in the serum and/or urine, along with various clinical parameters, such as the haemoglobin and serum calcium concentrations, the number of lytic bone lesions, and the presence or absence of renal failure. There are three stages according to Assessment of Tumour Mass.
Stage I: Low tumour mass
Stage II: Intermediate tumour mass
Stage III: High tumour mass

The stage of the disease at presentation is a strong determinant of survival, but has little influence on the choice of therapy since almost all patients have generalised disease (except for rare patients with solitary bone tumours or extramedullary plasmacytomas). Treatment option is influenced by the age and general health of the patient, prior therapy and the presence of complications of the disease.

Conventional chemotherapy produces remission rates of 50-70%, but in the majority patients relapse and tumor cells become refractory to the standard treatment. Treatment options range from pulse dexamethasone with or without thalidomide, conventional chemotherapy which is the combination of Melphalan and Predinisone, high-dose chemotherapy, and peripheral stem cell or allogeneic bone marrow transplantation.

However, virtually all patients succumb to this progressive disease. Median survival is of only three to four years and hasn't changed in the last two decades despite numerous treatment protocols.

Thus, as of today, there is no cure for multiple myeloma.

In addition, the presence of chromosome translocation (4; 14) is associated with a poor prognosis in MM patients. FGFR3 is a tyrosine kinase receptor which is not normally expressed in plasma cells and is therefore ectopically expressed as a result of the t (4; 14). FGFR3 has been shown to be an oncogene that can induce transformation in fibroblasts and that is inhibited by dominant negative inhibitors of the ras/MAPK pathway. It has also been shown to be transforming in hematopoeitic cells. These data validate FGFR3 as a potential target for experimental therapeutics in t-(4/14) MM.

In this particularly lethal form of MM, no current treatment has been able to induce long term remission.

Therefore, there is also a urgent need for a treatment being able to increase the response rate and survival of patients afflicted with MM, in particular MM expressing FGFR3 associated with the translocation (4; 14).

We have tested our compounds, 2-aminoarylthiazoles and 2-aminoaryloxazoles, hereinafter referred as the AB compounds, for which we filed WO 2004/014903 and WO 2005/040139.

We have found that AB compounds are non-cytotoxic anti-cancer agents which act as an inhibitor of the proto-oncogene c-kit. We report here that AB compounds are none only useful for treating MM in general but that they offer a unique dual inhibitory activity on c-kit and FGFR3, which properties are not found in any other tyrosine kinase inhibitor including STI 571.

Indeed, we have discovered that AB compounds inhibit phosphorylation of FGFR3 and display efficacy on multiple myeloma (4/14) expressing FGFR3. In fact, AB compounds block proliferation of MM cell lines expressing wild type or constitutively activated FGFR3. We also discovered a synergic effect of AB compounds and dexamethasone on MM and FGFR3+ myeloma.

Thus, we propose here a new route for treating MM and we provide for the first time a treatment for FGFR3+ myeloma, especially multiple myeloma with t-(4/14) translocations expressing FGFR3. The selective and potent effect of AB compounds is probably multifactorial and may include inhibition of cell proliferation, inhibition of cell cycle progression and the induction of apoptosis.

### DESCRIPTION

Therefore, the present invention is directed to a method for treating Multiple Myeloma (MM), FGFR3+ myeloma, especially relapsed or refractory multiple myeloma (4/14) expressing FGFR3, comprising administering a compound which is a dual C-KIT/FGFR3 inhibitor to a human in need of such treatment.

Such dual inhibitor is preferably chosen from compounds herein referred as the AB compounds: 2-aminoarylthiazoles and 2-aminoaryloxazoles (WO 2004/014903 and WO 2005/040139) incorporated herein by reference.

The above AB compounds block, with an IC50 reachable in vivo, the proliferation and survival of:
FGFR3 transfected cell lines,
Multiple myeloma cell lines that express constitutively FGFR3,
Multiple myeloma cell lines with constitutive activation FGFR3.

More particularly, these above compounds are useful for treating FGFR3+ myeloma. In addition, the invention benefits from the potency of AB compounds to act synergistically with dexamethasone to block proliferation and survival of multiple myeloma cell lines that express constitutively wild type or mutated FGFR3 with an IC50 reachable in vivo.

In this regard, the invention contemplates the combined use of a AB compound as defined above and dexamethasone for treating MM, especially FGFR3+ myeloma. It also relates to the combined use of AB compounds and current protocol, including vinca alkaloids, nitrosoureas, antracyclines and glucocorticoids and recent compounds such as thalidomide and bortezomib.

The present invention also relates to compounds belonging to the substituted thiazole and oxazole derivatives, especially 2-aminoarylthiazoles and 2-aminoaryloxazoles such as compounds of formula I. These compounds are capable of selectively inhibiting signal transduction involving the tyrosine phosphokinase c-kit, bcr-abl, Flt-3 and mutant forms thereof.

The above AB compounds block, with an IC50 reachable in vivo, the proliferation and survival of:
FGFR3 transfected cell lines,
Multiple myeloma cell lines that express constitutively FGFR3,
Multiple myeloma cell lines with constitutive activation FGFR3.

More particularly, these above compounds are useful for treating FGFR3+ myeloma.

In addition, the invention benefits from the potency of AB compounds to act synergistically with dexamethasone to block proliferation and survival of multiple myeloma cell lines that express constitutively wild type or mutated FGFR3 with an IC50 reachable in vivo.

In this regard, the invention contemplates a product for the combined administration of a AB compound as defined herein and dexamethasone for treating MM, especially FGFR3+ myeloma.

In a first embodiment preferred, the invention is aimed at compounds of formula I, which may represent either free base forms of the substances or pharmaceutically acceptable salts thereof:

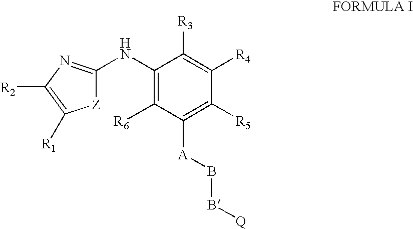

wherein substituents Z, A, B, B′, Q and R1-R6 in Formula I are defined as follows:
Z is oxygen or sulfur.
A and B′ is one of the following:
i) (R7)N(CH2)ₙ where n is 0 or 1
ii) O(CH2)ₙ where n is 0 or 1
iii) S(CH2)ₙ where n is 0 or 1
iv) (CH2)ₙ where n is 0, 1 or 2
v) C(O)(CH2)ₙ where n is 0 or 1
or when A and B′ each are a nitrogen, they may be taken together to form a bivalent radical of formula:

—(CH2)ₛ-X1-(CH2)ₜ- (a)

where s and t each independently is 1 or 2 and X1 being O, S, NR10, N[C(═O)R10] or (CH2)ₙ where n is 0 or 1, and wherein each hydrogen in said formula (a) may be substituted with halo or C₁₋₄alkyl.

B is one of the following:

### i) (R7)N

### ii) Oxygen

iii) S(O)ₙ where n is 0, 1 or 2

### iv) CH(R7)(R8)

v) C=δ, where δ is oxygen, sulfur, NH or N—CN

### vi) C(R7)=C(R8)

vii) N═C(R7)

R7 and R8 each independently are hydrogen, C₁₋₄alkyl, C₂₋₆alkenyl, C₂₋₆alkynyl, C₃₋₇cycloalkyl, C₁₋₄haloalkyl, C₁₋₄alkoxy, C₁₋₄hydroxyalkyl, C₁₋₄alkylamino.

R1 and R2 is selected from:

i) hydrogen, halogen (selected from F, Cl, Br or I), or
ii) an alkyl¹ group defined as a linear, branched or cycloalkyl group containing from 1 to 10 carbon atoms (such as for example from 2 to 4 or 1 to 5 or 1, 2, 3, 4, or 5 carbon atoms) and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl; as well as CO—R, COO—R, CONH—R, SO2-R, and SO2NH—R wherein R is a linear or branched alkyl group containing 1 to 10 carbon atoms and optionally substituted with at least one heteroatom, notably a halogen (selected from F, Cl, Br or I), oxygen, and nitrogen, the latter optionally in the form of a pendant basic nitrogen functionality; as well as a cycloalkyl or aryl¹ or heteroaryl¹ group optionally substituted by a pendant basic nitrogen functionality, or
iii) an aryl¹ group defined as phenyl or a substituted variant thereof bearing any combination, at any one ring position, of one or more substituents such as
Halogen (selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2-R or SO2NH—R or C(NOH)NH2, C(N)NH2 wherein R corresponds to hydrogen, alkyl¹, aryl or heteroaryl, or
   iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, benzoxazole, benzothiazole quinolinyl group, which may additionally bear any combination, at any one ring position, of one or more substituents such as
halogen (selected from F, Cl, Br or D;
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2—R or SO2NH—R wherein R corresponds to hydrogern, alkyl¹, or
   v) an O-aryl¹, or NH-aryl¹, or O-heteroaryl¹ or NH-heteroaryl¹ group
   vi) trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality, or
   vi) NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl¹ or heteroaryl¹.

R3, R4, R5 and R6 each independently are selected from hydrogen, halogen (selected from F, Cl, Br or I), a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, amino, C₁₋₆alkylamino, di(C₁₋₆alkyl)amino, carboxyl, cyano, nitro, formyl, hydroxy, and CO—R, COO—R, CONH—R, SO2-R, and SO2NH—R wherein R corresponds to hydrogen, alkyl¹, aryl or heteroaryl.

and wherein Q is selected from:

### i) Alkyl¹

### ii) Aryl¹

iii) Heteroaryl¹
as defined above.

Among the particular compounds of formula I, the invention is directed to compounds of the following formula II:

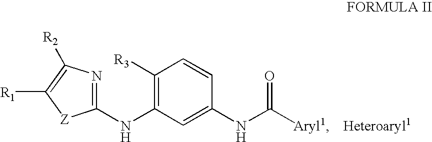

Z is oxygen or sulfur.

Aryl¹, Heteroaryl¹, R1, R2 and R3 have the meaning described above.

An example of preferred compounds of the above formula is depicted below:
001: N-{3-[5-(4-Cyano-phenyl)-oxazol-2-ylamino]-4-methyl-phenyl}-3-trifluoro methyl-benzamide

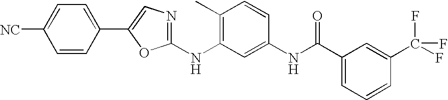

002: 4-(4-Methyl-piperazin-1-ylmethyl)-N-[4-methyl-3-(4-pyridin-3-yl-thiazol-2-ylamino)-phenyl]-benzamide

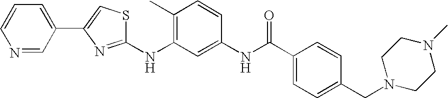

Among the particular compounds of formula I, the invention is directed to compounds of the following formula III:

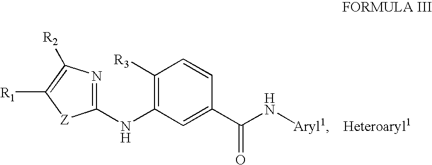

Z is oxygen or sulfur.

Aryl¹, Heteroaryl¹, R1, R2 and R3 have the meaning described above.

An example of preferred compounds of the above formula is depicted below:
003: N-(3-Chloro-phenyl)-4-methyl-3-(5-pyridin-4-yl-oxazol-2-ylamino)-benzamide

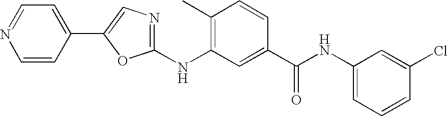

004: 4-Methyl-N-[4-(4-methyl-piperazin-1-ylmethyl)-phenyl]-3-(4-pyridin-3-yl-thiazol-2-ylamino)-benzamide

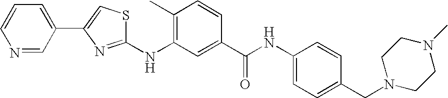

Among the particular compounds of formula I, the invention is directed to compounds of the following formula IV:

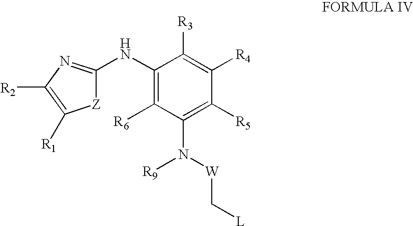

Wherein W is C═O or SO₂.

Z is oxygen or sulfur.

L is selected from Alkyl¹, Aryl¹ or Heteroaryl¹ as defined above.

R1, R2, R3, R4, R5 and R6 have the meaning described above. R9 is selected from hydrogen, a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; C₁₋₆alkyloxy, amino, hydroxyl.

An example of preferred compounds of the above formula is depicted below:
005: N-[4-Methyl-3-(5-pyridin-4-yl-oxazol-2-ylamino)-phenyl]-C-phenyl-methanesulfonamide

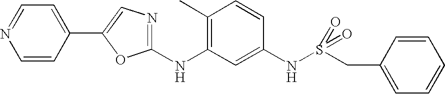

006: N-[4-Methyl-3-(4-pyridin-3-yl-thiazol-2-ylamino)-phenyl]-C-phenyl-methane sulfonamide

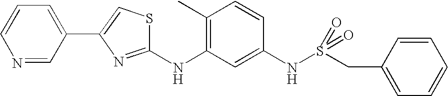

The compounds of the present invention may be prepared using the general protocols described in our previous applications WO 2004/014903 and WO 2005/040139.

In still another embodiment, the invention contemplates the method mentioned above, wherein said AB compound is selected from 2-(3-amino)arylamino-4-aryl-thiazoles such as those for which the applicant filed WO 2004/014903, incorporated herein in the description, especially compounds of formula V:

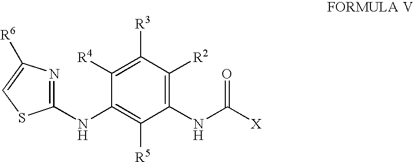

wherein X is R or NRR′ and wherein R and R′ are independently chosen from H, an aryl, a heteroaryl, an alkyl, or a cycloalkyl group optionally substituted with at least one heteroatom, such as for example a halogen chosen from F, I, Cl and Br and optionally bearing a pendant basic nitrogen functionality; or an aryl, a heteroaryl, an alkyl or a cycloalkyl group substituted with an aryl, a heteroaryl, an alkyl or a cycloalkyl group optionally substituted with at least one heteroatom, such as for example a halogen chosen from F, I, Cl and Br and optionally bearing a pendant basic nitrogen functionality,
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy;
R⁴ is hydrogen, halogen or a linear or branched alkyl group containing from 1 to 10 carbon atoms, trifluoromethyl or alkoxy;
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

Examples of group X include structures a to m shown below, wherein the wavy line or arrow corresponds to the point of attachment to core structure of formula V above:

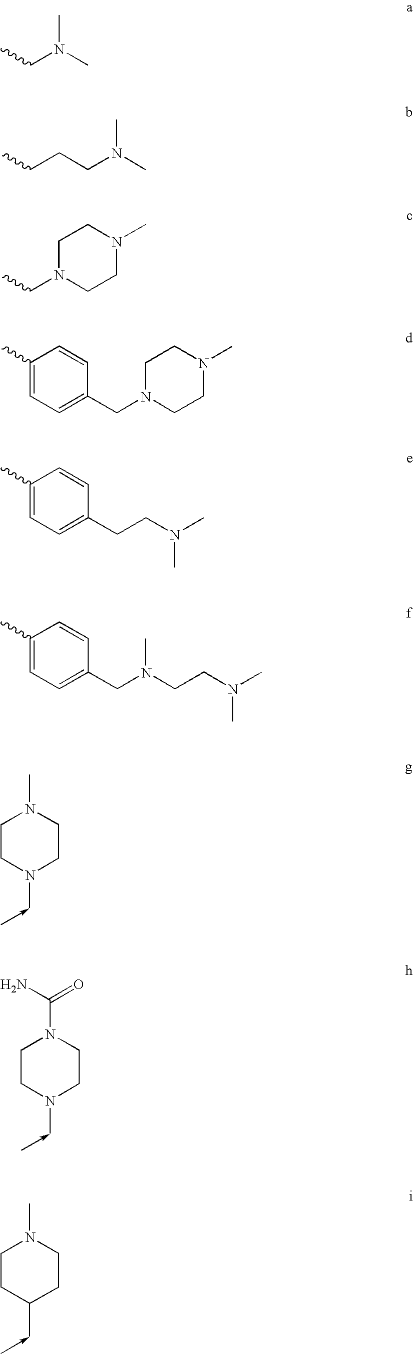

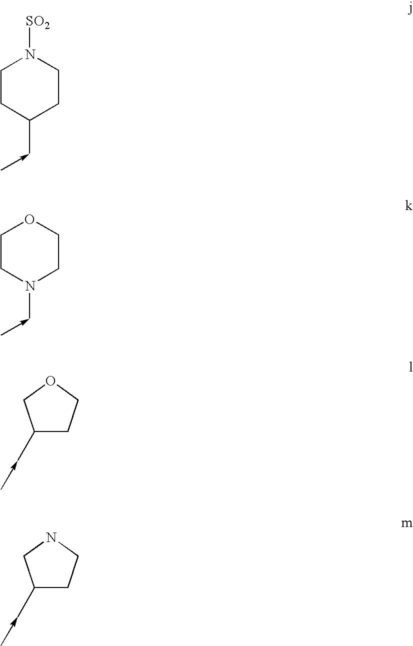

Among group a to f, is preferentially group d. Also, for g to m, the arrow may include a point of attachment to the core structure via a phenyl group.

In still another embodiment, the invention embraces the method as depicted above wherein said inhibitor is selected from compounds of Formula I, II, III, IV or V.

It also relates to the use of the compounds defined above to manufacture a medicament for treating Multiple Myeloma, FGFR3+ myeloma, especially relapsed or refractory multiple myeloma (4/14) expressing FGFR3.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

More particularly, the invention relates to a pharmaceutical composition intended for oral administration. Pharmaceutical compositions suitable for use in the invention include compositions wherein compounds for depleting mast cells, such as c-kit inhibitors, or compounds inhibiting mast cells degranulation are contained in an effective amount to achieve the intended purpose. The determination of an effective dose is well within the capability of those skilled in the art. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions which exhibit large therapeutic indices are preferred.

For example, MM and FGFR3+ myeloma are treated with daily administration of 40 mg, 100 mg, 200 mg, 400 mg or 800 mg of AB compound depending of the patient's weight. For example, from 6 to 8 mg/day/kg is administered to patients.

The invention also offers combined treatment with dexamethasone. In this regard, the invention is directed to a pharmaceutical composition comprising a AB compound as defined above and dexamethasone suitable for a simultaneous or separate administration over time. It also relates to a method for treating Multiple Myeloma (MM), FGFR3+ myeloma, especially relapsed or refractory multiple myeloma (4/14) expressing FGFR3, comprising administering a suitable amount of a AB compound as defined above and dexamethasone to a human in need of such treatment.

AB compounds may also be combined with pulse corticosteroids, autologous peripheral blood stream cells transplantation (enabling administration of high-dose melphalan), bortezomib, thalidomide and allogeneic stem cell transplantation.

### EXAMPLE 1

### Activity of AB Compounds

The action of AB compounds in inhibiting c-kit tyrosine kinase activity has been demonstrated in an ELISA assay using the purified intracellular soluble domain (567-976) of c-kit expressed in baculovirus measuring phosphorylation of a peptide target containing a tyrosine group. AB compounds potently inhibited enzymatic activity with an IC50 of below 0.1 μM. The specific anti-proliferative activity of AB compounds was exhibited in a selection of mammalian cell lines suitable for testing the specific activity of c-kit tyrosine kinase inhibitors with juxtamembrane mutations.

AB compounds are potent and selective inhibitors of c-kit (see Table I below) and also inhibit FGFR3 in vivo at IC50 below 2 μM.

TABLE I

| Enzyme | IC50 [μM] | Cell line | IC50 [μM] |
|---|---|---|---|
| c-Kit | below 0.1 | Ba/F3 Kit | 0.1 < IC50 < 1 |
| PDGF-beta | below 1 | Ba/F3 PDGFR | 0.1 < IC50 < 1 |
| FGFR3 | | LP1, NC1, OPM2 | <2 |
| ABL1 | below 10 | Ba/F3 p210Bcr-Abl | IC50 > 1 |
| VEGFR1 | IC50 > 100 | Ba/F3 IL3 | IC50 > 1 |
| EGFR | IC50 > 100 | Ba/F3 EGFR | IC50 > 1 |
| FGFR1 | IC50 > 100 | Ba/F3 RET | IC50 > 1 |
| FLT3 | IC50 > 100 | Ba/F3 TRKB | IC50 > 1 |
| JAK2 | IC50 > 100 | Ba/F3 FGFR1 | IC50 > 1 |
| AKT1 | below 100 | Ba/F3 FGFR3 | IC50 > 1 |
| PKC-alpha | about 100 | Ba/F3 FLT3 WT | IC50 > 1 |
| SRC | IC50 > 100 | Ba/F3 FLT3 ITD | IC50 > 1 |
| IGF1R | IC50 > 100 | Ba/F3 Tel-JaK1 | IC50 > 1 |
| PIM1 | below 50 | Ba/F3 Tel-JaK2 | IC50 > 1 |
| | | Ba/F3 Tel-JaK3 | IC50 > 1 |

AB compounds inhibit the proliferation of cells that express JM mutations of c-kit with an IC50 of less than 0.1 μM. The absence of non-specific cytotoxicity was demonstrated through proliferation of human T-lymphocyte populations, and of the Ba/F3 cell line in the presence of IL-3. The ability of AB compounds to induce apoptosis was demonstrated in a human mast cell line expressing the JMΔ27 mutated c-kit. In this experiment, after 48 hours, 0.1 μM AB1010 induced apoptosis of approximately 50% versus control cells in which 10% of cells were apoptotic. In addition, a separate cell line (Ba/F3-derived) expressing JMΔ27 was tested and apoptosis was induced to a level of approximately 85%.

### EXAMPLE 2

### Use of AB Compounds for Treating Myeloma Expressing FGFR3

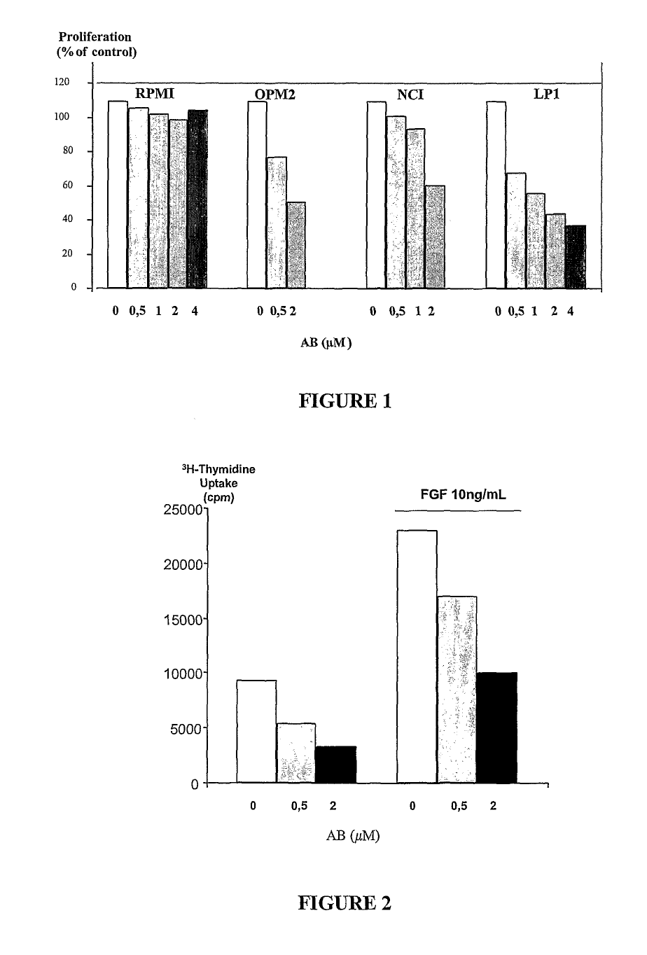

AB compounds are candidate for treating FGFR3+ myeloma since they inhibit the proliferation of multiple myeloma cell lines ectopically expressing FGFR3 (LP1 and NC1 human plasma cell line) or expressing constitutively activated FGFR3 (OPM2 human plasma cell line) (FIG. 1). FIG. 1 shows the inhibition of the proliferation (3H-Thymidine uptake) of human plasma cells lines expressing FGFR3 ectopically (LP1, NC1) or in a constitutively activated form (OPM2) by AB compound; control: FGFR3 negative plasma cell line RPMI.

The IC50 observed is reachable in vivo in humans. In vitro, synergy with dexamethasone was observed (data not shown).

In the presence of FGF, the proliferation of the t(4; 14) FGFR3+ human plasma cell line is enhanced, as measured by 3H-Thymidine uptake. AB1010 is able to inhibit the FGF-enhanced proliferation (FIG. 2). FIG. 2 shows the inhibition of the proliferation of the human plasma cell line LP1 expressing FGFR3 ectopically in the absence (left) of the presence of AB compound.

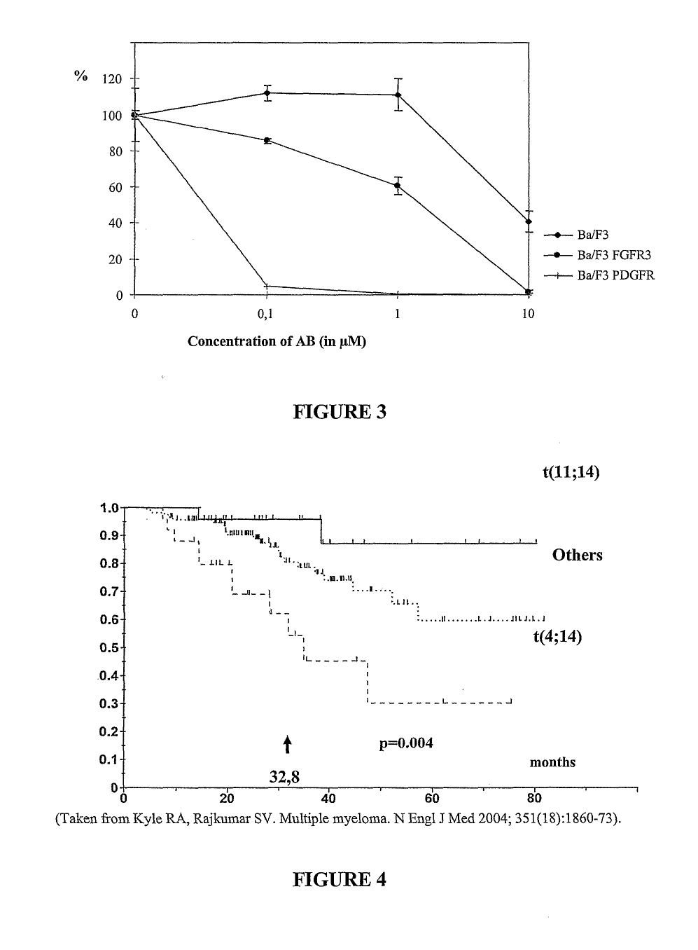

Furthermore, AB compound inhibit the proliferation of cell lines that have been transfected with the FGFR3 gene and over expressing FGFR3 with an IC50 that is reachable in vivo in humans (FIG. 3). FIG. 3 shows the inhibition of the proliferation of the human cell line Ba/F3 (losanges); Ba/F3 transfected with FGFR3 gene (circles) and Ba/F3 transfected with the PDGFR gene, in the presence of AB compound.

In MM, higher risk is associated with a t(4;14) translocation in malignant plasma cells. In presence of the t(4;14) translocation, the prognosis is extremely poor (FIG. 4), especially when patient relapse. No current treatment, including investigational use of allogenic stem cells transplantation, is able to induce long term remission. Those latter patients with no therapeutic options and aggressive disease are a primary population that would benefit AB compounds efficacy.

1. A method for treating Multiple Myeloma, FGFR3+ myeloma, especially relapsed or refractory multiple myeloma (4/14) expressing FGFR3, comprising administering a dual C-KIT/FGFR3 inhibitor to a human in need of such treatment.

2. The method according to claim 1 or 2 wherein said inhibitor is selected from the group consisting of 2-aminoarylthiazoles and 2-aminoaryloxazoles.

3. The method according to claim 2, wherein said inhibitor is selected from compounds of formula I: 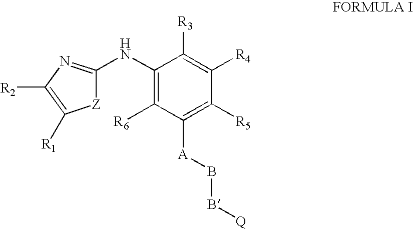
wherein substituents Z, A, B, B′, Q and R1-R6 in Formula I are defined as follows:
Z is oxygen or sulfur.
A and B′ is one of the following:
i) (R7)N(CH2)ₙ where n is 0 or 1
ii) O(CH2)ₙ where n is 0 or 1
iii) S(CH2)ₙ where n is 0 or 1
iv) (CH2)ₙ where n is 0, 1 or 2
v) C(O)(CH2)ₙ where n is 0 or 1
or when A and B′ each are a nitrogen, they may be taken together to form a bivalent radical of formula:
—(CH2)ₛ-X1-(CH2)ₜ- (a)
where s and t each independently is 1 or 2 and X1 being O, S, NR10, N[C(═O)R10] or (CH2)ₙ where n is 0 or 1, and wherein each hydrogen in said formula (a) may be substituted with halo or C₁₋₄alkyl.
B is one of the following:
i) (R7)N
ii) Oxygen
iii) S(O)ₙ where n is 0, 1 or 2
iv) CH(R7)(R8)
v) C=δ, where δ is oxygen, sulfur, NH or N—CN
vi) C(R7)=C(R8)
vii) N═C(R7)
R7 and R8 each independently are hydrogen, C₁₋₄alkyl, C₂₋₆alkenyl, C₂₋₆alkynyl, C₃₋₇cycloalkyl, C₁₋₄haloalkyl, C₁₋₄alkoxy, C₁₋₄hydroxyalkyl, C₁₋₄alkylamino.
R1 and R2 is selected from:
i) hydrogen, halogen (selected from F, Cl, Br or I), or
ii) an alkyl¹ group defined as a linear, branched or cycloalkyl group containing from 1 to 10 carbon atoms and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl; as well as CO—R, COO—R, CONH—R, SO2-R, and SO2NH—R wherein R is a linear or branched alkyl group containing 1 to 10 carbon atoms and optionally substituted with at least one heteroatom, notably a halogen (selected from F, Cl, Br or I), oxygen, and nitrogen, the latter optionally in the form of a pendant basic nitrogen functionality; as well as a cycloalkyl or aryl¹ or heteroaryl¹ group optionally substituted by a pendant basic nitrogen functionality, or
iii) an aryl¹ group defined as phenyl or a substituted variant thereof bearing any combination, at any one ring position, of one or more substituents such as
Halogen (selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2-R or SO2NH—R or C(NOH)NH2, C(N)NH2 wherein R corresponds to hydrogen, alkyl¹, aryl or heteroaryl, or
iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, benzoxazole, benzothiazole quinolinyl group, which may additionally bear any combination, at any one ring position, of one or more substituents such as
halogen (selected from F, Cl, Br or I);
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2—R or SO2NH—R wherein R corresponds to hydrogern, alkyl¹, or
v) an O-aryl¹, or NH-aryl¹, or O-heteroaryl¹ or NH-heteroaryl¹ group
vi) trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality, or
vi) NHCO—R or NHCOO—R or NHCONH—R or NHSO2-R or NHSO2NH—R or CO—R or COO—R or CONH—R or SO2—R or SO2NH—R wherein R corresponds to hydrogen, alkyl¹, aryl¹ or heteroaryl¹.
R3, R4, R5 and R6 each independently are selected from hydrogen, halogen (selected from F, Cl, Br or I), a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, amino, C₁₋₆alkylamino, di(C₁₋₆alkyl)amino, carboxyl, cyano, nitro, formyl, hydroxy, and CO—R, COO—R, CONH—R, SO2—R, and SO2NH—R wherein R corresponds to hydrogen, alkyl¹, aryl or heteroaryl.
and wherein Q is selected from:
i) Alkyl¹
ii) Aryl¹
iii) Heteroaryl¹
as defined above.

4. The method according to claim 3, wherein said inhibitor is selected from compounds of formula II: 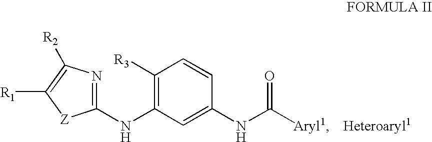
Z is oxygen or sulfur.
Aryl¹, Heteroaryl¹, R1, R2 and R3 have the meaning described above.

5. The method according to claim 3, wherein said inhibitor is selected from compounds of formula III: 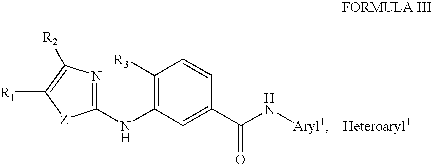
Z is oxygen or sulfur.
Aryl¹, Heteroaryl¹, R1, R2 and R3 have the meaning described above.

6. The method according to claim 3, wherein said inhibitor is selected from compounds of formula IV: 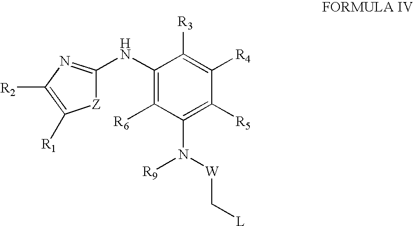
Wherein W is C═O or SO₂.
Z is oxygen or sulfur.
L is selected from Alkyl¹, Aryl¹ or Heteroaryl¹ as defined above.
R1, R2, R3, R4, R5 and R6 have the meaning described above.
R9 is selected from hydrogen, a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; C₁₋₆alkyloxy, amino, hydroxyl.

7. A method according to one of claims 1 to 6, wherein said inhibitor is:
N-{3-[5-(4-Cyano-phenyl)-oxazol-2-ylamino]-4-methyl-phenyl}-3-trifluoromethyl-benzamide
4-(4-Methyl-piperazin-1-ylmethyl)-N-[4-methyl-3-(4-pyridin-3-yl-thiazol-2-ylamino)-phenyl]-benzamide
N-(3-Chloro-phenyl)-4-methyl-3-(5-pyridin-4-yl-oxazol-2-ylamino)-benzamide
4-Methyl-N-[4-(4-methyl-piperazin-1-ylmethyl)-phenyl]-3-(4-pyridin-3-yl-thiazol-2-ylamino)-benzamide
N-[4-Methyl-3-(5-pyridin-4-yl-oxazol-2-ylamino)-phenyl]-C-phenyl-methanesulfonamide
N-[4-Methyl-3-(4-pyridin-3-yl-thiazol-2-ylamino)-phenyl]-C-phenyl-methane sulfonamide

8. The use of a compound as defined in one of claims 1 to 7 to manufacture a medicament for treating Multiple Myeloma such as FGFR3+ myeloma.

9. The use of a compound as defined in one of claims 1 to 7 to manufacture a medicament for treating relapsed or refractory multiple myeloma (4/14) expressing FGFR3.

10. A pharmaceutical composition comprising a compound as defined in one of claims 1 to 7 and dexamethasone suitable for a simultaneous or separate administration over time.

11. A method for treating Multiple Myeloma (MM), FGFR3+ myeloma, especially relapsed or refractory multiple myeloma (4/14) expressing FGFR3, comprising administering a suitable amount of a compound as defined in one of claims 1 to 7 and dexamethasone to a human in need of such treatment.
